(19) United States
(12) Patent Application Publication
(10) Pub. No.: US 2018/0271360 A1
(43) Pub. Date: Sep. 27, 2018

(54) METHOD FOR DETERMINING A RISK SCORE OF AN EYE DISEASE FOR A USER AND SYSTEM FOR THE EXECUTION OF SUCH METHOD

(71) Applicant: Essilor International, Charenton-Le-Pont (FR)

(72) Inventors: Coralie BARRAU, Charenton-Le-Pont (FR); Thierry Villette, Charenton-Le-Pont (FR)

(73) Assignee: Essilor International, Charenton-Le-Pont (FR)

(21) Appl. No.: 15/928,619

(22) Filed: Mar. 22, 2018

(30) Foreign Application Priority Data
Mar. 23, 2017 (EP) 17305330.7

(51) Int. Cl. A61B 3/00; A61B 3/11; A61B 3/06; A61B 5/00; A61B 5/16; A61B 3/14

(52) U.S. Cl. CPC A61B 3/0025; A61B 3/112; A61B 3/063; A61B 2576/02; A61B 5/161; A61B 3/066; A61B 3/14; A61B 5/7275

(57) ABSTRACT

A method, implemented by a computer device, for determining a risk score of an eye disease for a user, the method including a first eye-mediated physiological parameter providing step, during which a first eye-mediated physiological parameter indicative of a first eye-mediated perception or behaviour of the user is provided, a second eye-mediated physiological parameter providing step, during which a second eye-mediated physiological parameter indicative of a second eye-mediated perception or behaviour of the user is provided, and a risk score determining step, during which the risk score of eye disease is determined based on the first eye-mediated physiological parameter and on the second eye-mediated physiological parameter.

### FIELD OF THE INVENTION

The invention relates to a method, implemented by computer means, for determining a risk score of an eye disease for a user.

The invention further relates to a system for the execution of the method for determining a risk score of an eye disease for a user and to a program adapted to perform the method for determining a risk score of an eye disease for a user when installed and executed in the system.

### BACKGROUND OF THE INVENTION

Some eye diseases, for instance age-related macular degeneration (“AMD”), cause irreversible blindness of the persons having the eye disease.

For example, AMD is often diagnosed in an advanced stage. Most of the persons having an AMD are unaware that they have AMD until they are diagnosed with late-stage. Moreover, most of the persons having an AMD, when first diagnosed, already have an irreversible and significant loss of visual acuity. For diagnosed persons, behavior modification, nutritional supplementation, photo-protection, anti-vascular endothelial growth factor (“VEGF”) treatment may be used to reduce the incidence of progression of irreversible visual loss.

Since some eye diseases progress quickly, there is a need for an early detection of these eye diseases. Early detection of eye diseases may offer the opportunity for early treatment and better possibilities for visual outcomes.

The most common approach in assessing retinal function for diagnosing AMD is visual acuity. However, the acuity testing is simple and quick, no information on retinal function in early AMD are provided, since the visual acuity changes due to early AMD are undetectable.

Hence, early eye disease, such as AMD, cannot be detected in daily life and during routine vision exams.

Therefore, there is a need for a method for detecting early eye disease, and more precisely for assessing functional change in early eye disease to better monitor its progression.

One object of the invention is to provide such a method.

### SUMMARY OF THE INVENTION

To this end, the invention proposes a method implemented by computer means, for determining a risk score of an eye disease for a user, the method comprising:
a first eye-mediated physiological parameter providing step, during which a first eye-mediated physiological parameter indicative of a first eye-mediated perception or behaviour of the user is provided,
a second eye-mediated physiological parameter providing step, during which a second eye-mediated physiological parameter indicative of a second eye-mediated perception or behaviour of the user is provided, and
a risk score determining step, during which the risk score of eye disease is determined based on the first eye-mediated physiological parameter and on the second eye-mediated physiological parameter.

Advantageously, the method of the invention allows calculating the early risk score of an eye disease based on multiple parameters. More precisely, the method of the invention allows detecting the first functional signs of early eye disease to establish a relevant and updatable risk score based on different parameters.

According to embodiments, the method for providing a set of data according to the invention may further comprise one or several of the following features according to any possible combination:
the first eye-mediated physiological parameter relates to the dark adaptation of at least one eye of the user, and wherein the second eye-mediated physiological parameter relates to the pupil light reflex of at least one eye of the user and/or to the flicker sensitivity of at least one eye of the user; and/or
the first eye-mediated physiological parameter provided during the first eye-mediated physiological parameter providing step and/or the second eye-mediated physiological parameter provided during the second eye-mediated physiological parameter providing step are determined by at least one test chosen among the list of tests consisting of:
   determination of the transient pupil response;
   determination of the latency to constriction;
   determination of the constriction amplitude;
   determination of the post illumination state;
   determination of the pupil diameter after light offset;
   determination of the phasic pupil response;
   determination of the rod recovery on dark adaptation;
   determination of the cone recovery on dark adaptation;
   determination of the flicker detection threshold; and/or
the method further comprises a third eye-mediated physiological parameter providing step, during which a third eye-mediated physiological parameter indicative of a third eye-mediated perception or behaviour of the user is provided, wherein the risk score of an eye disease is determined considering the third eye-mediated physiological parameter; and/or
the third eye-mediated physiological parameter relates to the photosensitivity of at least one eye of the user and/or the color sensitivity of at least one eye of the user and/or the contrast sensitivity of at least one eye of the user and/or the glare recovery of at least one eye of the user and/or the shape deformation sensitivity of at least one eye of the user and/or the visual acuity of at least one eye of the user and/or the spatial contrast sensitivity of at least one eye of the user and/or the macular pigment density of at least one eye of the user; and/or
the third eye-mediated physiological parameter provided during the third eye-mediated physiological parameter providing step is determined by at least one test chosen among the list of tests consisting of:
   determination of the visual acuity;
   determination of the photosensitivity threshold;
   determination of the cone contrast threshold;
   determination of the cone recovery on glare recovery; and/or
the method further comprises a life profile data providing step, during which life profile data relative to at least one parameter of the life profile of the user is provided, wherein the risk score of an eye disease is determined considering the life profile data; and/or
the parameter of the life profile of the user relates to the age of the user and/or the gender of the user and/or the professional situation of the user and/or the personal situation of the user and/or the living place of the user and/or the general state of health of the user and/or the health history of the user and/or the health history of the family of the user and/or the food habits of the user and/or the physical activity habits of the user and/or the rhythm of life of the user; and/or
the method further comprises an environment parameter providing step, during which an environment parameter indicative of the environment of the user is provided, wherein the risk score of an eye disease is determined considering the environment parameter; and/or
the parameter of the environment of the user relates to the light exposure of the user and/or the radiance and/or the spectral emission and/or spatial distribution and/or history of exposure of the light received by the user; and/or
the method further comprises an eye imaging parameter provided step, during which an eye imaging parameter indicative of an eye imaging of the at least one eye of the user is provided, wherein the risk score of an eye disease is determined considering the eye imaging parameter; and/or
the eye imaging parameter relates to at least a feature of a drusen of an eye of the wearer, said feature comprising at least one of the size and shape of the drusen, the total area of the druses and the location of the drusen; and/or
the method further comprises an updating step, during which the first eye-mediated physiological parameter and/or the second eye-mediated physiological parameter and/or the third eye-mediated physiological parameter and/or the life profile data and/or the environment data and/or the eye imaging parameter are updated, wherein the risk score of an eye disease is determined considering the updated data.

Advantageously, the method of the invention allows updating the early risk score of an eye disease based on multiple parameters. In other words, the method of the invention allows providing to the user an accurate and updated value of the risk score.

Advantageously, the method of the invention allows helping the user to be aware of the effect of different parameters on his risk score and having the user either changing his habits or on the contrary amplifying some behaviour or habits.

The invention further relates to a system for the execution of the method for determining a risk score of an eye disease for a user according to the invention.

The invention further relates to a program adapted to perform the method for determining a risk score of an eye disease for a user according to the invention when installed and executed in the system of the invention.

The invention further relates to a system for determining a risk score of an eye disease for a user, the system comprising:
a first eye-mediated physiological parameter providing means for providing a first eye-mediated physiological parameter indicative of a first eye-mediated perception or behaviour of the user,
a second eye-mediated physiological parameter providing means for providing a second eye-mediated physiological parameter indicative of a second eye-mediated perception or behaviour of the user, and
a risk score determining means for determining the risk score of eye disease based on the first eye-mediated physiological parameter and on the second eye-mediated physiological parameter.

The invention further relates to a computer program product comprising one or more stored sequences of instructions that are accessible to a processor and which, when executed by the processor, causes the processor to carry out the steps of the method according to the invention.

The invention further relates to a system for determining a risk score of an eye disease for a user comprising a processor adapted to store one or more sequences of instruction and to carry out at least one of the steps of the method according to the invention.

The invention further relates to a computer readable storage medium having a program recorded thereon, where the program makes the computer execute the steps of the method according to the invention.

### BRIEF DESCRIPTION OF THE DRAWINGS

Other characteristics and advantages of the invention will become more apparent from the claims and from the following description of some embodiments given by way of example without limitation with reference to the drawing:

FIG. 1 is a flowchart of the different steps of a method for determining a risk score of an eye disease for a user according to the invention.

### DETAILED DESCRIPTION OF PREFERRED EMBODIMENTS

The invention relates to a method, for example implemented by computer means, for determining a risk score of an eye disease for a user.

For instance, the method allows determining a risk score of age-related macular degeneration.

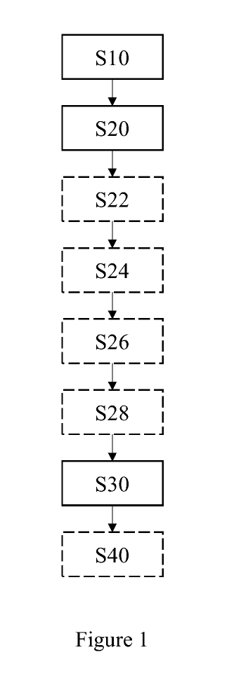

A flowchart of the different steps of the method for determining a risk score of an eye disease for a user according to the invention is represented in FIG. 1.

The method comprises a first eye-mediated physiological parameter providing step (S10) and a second eye-mediated physiological parameter providing step (S20).

During the first eye-mediated physiological parameter providing step (S10), a first eye-mediated physiological parameter indicative of a first eye-mediated perception or behaviour of the user is provided.

The first eye-mediated physiological parameter may relate to the dark adaptation of at least one eye of the user.

During the second eye-mediated physiological parameter providing step (S20), a second eye-mediated physiological parameter indicative of a second eye-mediated perception or behaviour of the user is provided.

The second eye-mediated physiological parameter may relate to the pupil light reflex of at least one eye of the user and/or to the flicker sensitivity of at least one eye of the user.

The first eye-mediated physiological parameter provided during the first eye-mediated physiological parameter providing step (S10) may be determined by at least one test. Likewise, the second eye-mediated physiological parameter provided during the second eye-mediated physiological parameter providing step (S20) may be determined by at least one test.

The tests for determining the first and/or second eye-mediated physiological parameters may be chosen among the list of tests consisting of the determination of the transient pupil response, the determination of the latency to constriction, the determination of the constriction amplitude, the determination of the post illumination state, the determination of the pupil diameter after light offset, the determination of the phasic pupil response, the determination of the rod recovery on dark adaptation, the determination of the cone recovery on dark adaptation, the determination of the flicker detection threshold.

The dark adaptation may be determined using measurements of the rod recovery on dark adaptation or of the cone recovery on dark adaptation. This test is reproducible and has a good diagnostic capacity.

The tests for determining the dark adaptation of at least one eye of the user may be performed with low light levels.

The pupil light reflex may be determined using pupil recordings with sinusoidal light excitations, for instance at around 480 nm, namely at high excitation of the intrinsically photosensitive retinal ganglion cells (“ipRGC”) and at around 630 nm, or with sinusoidal light stimulus, for instance 11.9 seconds sinusoidal stimulus. The pupil light reflex may be determined a priori to the illumination and after the illumination. The pupil light reflex may be determined during different duration of stimulation. The pupil light reflex may be determined using multifocal pupillography or irradiances bellow and above the melanopsin threshold to detect both deficits in rods and cones functions and deficits in ipRGC function. This test is quick, objective, non-invasive and reproducible.

The flicker sensitivity may be determined using a flickering stimulus. This test is quick, for instance around 4 to 7 minutes, reproducible, clinically applicable and has a good diagnostic capacity.

The first and second eye-mediated physiological parameters may be static parameters or dynamic parameters. Moreover, one among the first and second eye-mediated physiological parameters may be a static parameter, and the other among the first and second eye-mediated physiological parameters may be dynamic parameter.

The method comprises a risk score determining step (S30), during which the risk score of eye disease is determined based on the first eye-mediated physiological parameter and on the second eye-mediated physiological parameter.

Advantageously, the determination of the risk score of eye disease based on a plurality of parameters allows improving the early detection of eye disease.

The risk score of eye disease may be determined based on a combination of static and dynamic parameters.

The method may comprise a third eye-mediated physiological parameter providing step (S22), during which a third eye-mediated physiological parameter indicative of a third eye-mediated perception or behaviour of the user is provided.

The third eye-mediated physiological parameter may relate to the photosensitivity of at least one eye of the user and/or the color sensitivity of at least one eye of the user and/or the contrast sensitivity of at least one eye of the user and/or the shape deformation sensitivity of at least one eye of the user and/or the visual acuity of at least one eye of the user and/or the spatial contrast sensitivity of at least one eye of the user and/or the macular pigment density of at least one eye of the user.

The third eye-mediated physiological parameter may relate to the glare recovery of at least one eye of the user and/or to the photo-stress recovery of at least one eye of the user.

The third eye-mediated physiological parameter may relate to the chromatic function of at least one eye of the user and/or the visual distortion of at least one eye of the user and/or the low contrast visual acuity of at least one eye of the user.

The third eye-mediated physiological parameter provided during the third eye-mediated physiological parameter providing step (S22) may be determined by at least one test.

The tests for determining the third eye-mediated physiological parameter may be chosen among the list of tests consisting of: the determination of the visual acuity, the determination of the photosensitivity threshold, the determination of the cone contrast threshold, the determination of the cone recovery on glare recovery.

The list of tests for determining the third eye-mediated physiological parameter may comprise the determination of the colour sensitivity threshold. This test is fast and user-friendly.

The list of tests for determining the third eye-mediated physiological parameter may comprise the determination of the photo-stress recovery and/or the determination of the glare recovery. These tests are fast and user-friendly.

The photosensitivity threshold may be determined during dynamic automatized light sequences, for instance with 10 seconds per steps, 50 lux step from 0 to 1000 lux, or 100 lux step from 1000 to 2000 lux, or 200 lux step from 2000 to 4000 lux, or 400 lux step from 4000 to 8000 lux. The photosensitivity threshold may be determined during random fixed light levels sequences. The photosensitivity threshold may be determined using a portative and uniform lighting sphere, or using ultra-violet (“UV”) light-emitting diodes (“LED”) for progressive activation of photochromic lenses. The photosensitivity threshold may be determined using correlated colour-temperature (“CCT”) from a natural white light, for instance 4000 K, to a very cold white light, for instance greater than 6500 K. This test is easy to implement and clinically applicable.

The contrast sensitivity may be determined using a cone contrast threshold.

The visual distortion may be determined using Amsler grid test.

The third eye-mediated physiological parameter may be a static parameter or a dynamic parameter.

The risk score of an eye disease may be determined considering the third eye-mediated physiological parameter.

The method may comprise a life profile data providing step (S24), during which life profile data relative to at least one parameter of the life profile of the user is provided.

The parameter of the life profile of the user may relate to the age of the user and/or the gender of the user and/or the professional situation of the user and/or the personal situation of the user and/or the living place of the user and/or the general state of health of the user and/or the physical activity habits of the user.

The parameter of the life profile of the user may relate to the health history of the user, for instance to photosensitive treatments or to cataract surgery of at least one eye of the user.

The parameter of the life profile of the user may relate to the ethnicity of the user.

The parameter of the life profile of the user may relate to the health history of the family of the user, for example if parents of the user have an eye disease, such as AMD. The parameter of the life profile of the user may relate to the genetics of the user.

The parameter of the life profile of the user may relate to the food habits of the user and/or the rhythm of life of the user, for instance if the user smokes or the diet of the user.

The parameter of the life profile of the user may relate to oxidative stressors, such as a smoking treatment, a diet poor in antioxidants, a light treatment or a photosensitive treatment.

The parameter of the life profile of the user may relate to the protections used by the user, namely if the user uses sunglasses or hats.

The parameter of the life profile of the user may be determined with a questionnaire.

The risk score of an eye disease may be determined considering the life profile data.

The method may comprise an environment parameter providing step (S26), during which an environment parameter indicative of the environment of the user is provided.

The parameter of the environment of the user may relate to the light exposure of the user and/or the radiance and/or the spectral emission and/or spatial distribution and/or history of exposure of the light received by the user.

The light exposure of the user may be defined by the correlation of the number of light sources and/or the localization of the light sources and/or the spatial distribution of the light sources and/or the radiance of the light sources including the directivity of the light and/or the spectral distribution of the light sources and/or the exposure duration of the user and/or the repetition of the exposure of the user.

The light exposure of the user may be determined with a connected eyewear comprising spectrometers and/or colored photodiodes, such as UV, blue, green, red, and infra-red (“IR”) photodiodes.

The risk score of an eye disease may be determined considering the environment parameter.

The method may comprise an eye imaging parameter provided step (S28), during which an eye imaging parameter indicative of an eye imaging of the at least one eye of the user is provided.

The eye imaging parameter may relate to at least a feature of a drusen of an eye of the wearer. The feature of a drusen of an eye of the wearer may comprise at least one of the size and shape of the drusen, the total area of the druses and the location of the drusen.

The feature of a drusen of an eye of the wearer may comprise the type of the drusen, for instance a hard type drusen, when the size of the drusen is smaller than 63 μm, an intermediate soft type drusen, when the size of the drusen is comprised between 63 μm and 125 μm, a large semi-solid type drusen, when the size of the drusen is greater than 125 μm.

The eye imaging parameter may relate to pigmentary abnormalities of at least one eye of the user and/or a geographic atrophy of at least one eye of the user and/or a choroidal neovascularization of at least one eye of the user.

The risk score of an eye disease may be determined considering the eye imaging parameter.

In particular, an age-related eye disease study risk (“AREDS”) may be only based on an eye imaging parameter. For instance, the AREDS may comprise scores between 1 and 4. The score 1 corresponds to no drusen in at least one eye of the user or the size of the drusen is smaller than 63 μm and the total area of the druses is smaller than 125 μm. The score 2 corresponds to no geographic atrophy and the size of the drusen is greater than or equal to 63 μm and smaller than 125 μm and the total area of the druses is greater than or equal to 125 μm. The score 3a corresponds to soft distinct drusen, the size of the drusen is greater than or equal to 63 μm and smaller than 125 μm and the total area of the druses is greater than 360 μm. The score 3b corresponds to soft indistinct drusen, the size of the drusen is greater than or equal to 63 μm and smaller than 125 μm and the total area of the druses is greater than or equal to 656 μm. The score 4 corresponds to geographic atrophy or choroidal neovascularization and a visual acuity smaller than 20/32.

The method may further comprise an updating step (S40).

During the updating step (S40), the first eye-mediated physiological parameter and/or the second eye-mediated physiological parameter and/or the third eye-mediated physiological parameter and/or the life profile data and/or the environment data and/or the eye imaging parameter are updated.

The risk score of an eye disease may be determined considering the updated data.

Advantageously, the updating of the risk score of an eye disease of a user allows providing to the user an accurate and updated value of the risk score. More precisely, this updating allows helping the user to be aware of the effect of different parameters on his risk score and having the user either changing his habits or on the contrary amplifying some behaviour or habits.

The first eye-mediated physiological parameter and/or the second eye-mediated physiological parameter and/or the third eye-mediated physiological parameter and/or the life profile data and/or the environment data and/or the eye imaging parameter may be personalized according to the user and/or to the activities of the user and/or to the goals and performances to be reached by the user and/or to the user's doctor and/or to the user's localization.

The invention further relates to a system for the execution of the method for determining a risk score of an eye disease for a user as described previously.

The system may comprise a sensor or a plurality of sensors for measuring the first eye-mediated physiological parameter and/or the second eye-mediated physiological parameter and/or the third eye-mediated physiological parameter and/or the environment data and/or the eye imaging parameter.

After determining the risk score of an eye disease for a user, the system may activate specific functions depending on the risk score determined. For instance, a specific function to be activated may be an automation of home automation or of a vehicle, or the trigger of an alarm at the medical centre of the user.

The invention has been described above with the aid of embodiments without limitation of the general inventive concept. Moreover, the embodiments of the invention may be combined without any restriction.

Many further modifications and variations will suggest themselves to those skilled in the art upon making reference to the foregoing illustrative embodiments, which are given by way of example only and which are not intended to limit the scope of the invention, that being determined solely by the appended claims.

In the claims, the word “comprising” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the invention.

1. A method, implemented by computer, for determining a risk score of an eye disease for a user, the method comprising:
a first eye-mediated physiological parameter providing step, during which a first eye-mediated physiological parameter indicative of a first eye-mediated perception or behaviour of the user is provided,
a second eye-mediated physiological parameter providing step, during which a second eye-mediated physiological parameter indicative of a second eye-mediated perception or behaviour of the user is provided, and
a risk score determining step, during which the risk score of eye disease is determined based on the first eye-mediated physiological parameter and on the second eye-mediated physiological parameter.

2. The method according to claim 1, wherein the first eye-mediated physiological parameter relates to the dark adaptation of at least one eye of the user, and wherein the second eye-mediated physiological parameter relates to the pupil light reflex of at least one eye of the user and/or to the flicker sensitivity of at least one eye of the user.

3. The method according to claim 1, wherein the first eye-mediated physiological parameter provided during the first eye-mediated physiological parameter providing step and/or the second eye-mediated physiological parameter provided during the second eye-mediated physiological parameter providing step are determined by at least one test chosen among the list of tests consisting of:
determination of the transient pupil response;
determination of the latency to constriction;
determination of the constriction amplitude;
determination of the post illumination state;
determination of the pupil diameter after light offset;
determination of the phasic pupil response;
determination of the rod recovery on dark adaptation;
determination of the cone recovery on dark adaptation;
determination of the flicker detection threshold.

4. The method according to claim 1, further comprising:
a third eye-mediated physiological parameter providing step, during which a third eye-mediated physiological parameter indicative of a third eye-mediated perception or behaviour of the user is provided,
wherein the risk score of an eye disease is determined considering the third eye-mediated physiological parameter.

5. The method according to claim 4, wherein the third eye-mediated physiological parameter relates to the photosensitivity of at least one eye of the user and/or the color sensitivity of at least one eye of the user and/or the contrast sensitivity of at least one eye of the user and/or the glare recovery of at least one eye of the user and/or the shape deformation sensitivity of at least one eye of the user and/or the visual acuity of at least one eye of the user and/or the spatial contrast sensitivity of at least one eye of the user and/or the macular pigment density of at least one eye of the user.

6. The method according to claim 4 wherein the third eye-mediated physiological parameter provided during the third eye-mediated physiological parameter providing step is determined by at least one test chosen among the list of tests consisting of:
determination of the visual acuity;
determination of the photosensitivity threshold;
determination of the cone contrast threshold;
determination of the cone recovery on glare recovery.

7. The method according to claim 1, further comprising:
a life profile data providing step, during which life profile data relative to at least one parameter of the life profile of the user is provided,
wherein the risk score of an eye disease is determined considering the life profile data.

8. The method according to claim 7, wherein the parameter of the life profile of the user relates to the age of the user and/or the gender of the user and/or the professional situation of the user and/or the personal situation of the user and/or the living place of the user and/or the general state of health of the user and/or the health history of the user and/or the health history of the family of the user and/or the food habits of the user and/or the physical activity habits of the user and/or the rhythm of life of the user.

9. The method according to claim 1, further comprising:
an environment parameter providing step, during which an environment parameter indicative of the environment of the user is provided,
wherein the risk score of an eye disease is determined considering the environment parameter.

10. The method according to claim 9, wherein the parameter of the environment of the user relates to the light exposure of the user and/or the radiance and/or the spectral emission and/or spatial distribution and/or history of exposure of the light received by the user.

11. The method according to claim 1, further comprising:
an eye imaging parameter provided step, during which an eye imaging parameter indicative of an eye imaging of the at least one eye of the user is provided,
wherein the risk score of an eye disease is determined considering the eye imaging parameter.

12. The method according to claim 11, wherein the eye imaging parameter relates to at least a feature of a drusen of an eye of the wearer, said feature comprising at least one of the size and shape of the drusen, the total area of the druses and the location of the drusen.

13. The method according to claim 1, further comprising:
an updating step, during which the first eye-mediated physiological parameter and/or the second eye-mediated physiological parameter and/or the third eye-mediated physiological parameter and/or the life profile data and/or the environment data and/or the eye imaging parameter are updated,
wherein the risk score of an eye disease is determined considering the updated data.

14. A system for the execution of the method for determining a risk score of an eye disease for a user according to claim 1.

15. A program adapted to perform the method for determining a risk score of an eye disease for a user according to claim 1 when installed and executed in the system.
